(19) United States
(12) Patent Application Publication
(10) Pub. No.: US 2006/0160197 A1
(43) Pub. Date: Jul. 20, 2006

(54) Method for the production of glycolic acid from ammonium glycolate by solvent extraction

(76) Inventors: Xu Li, Newark, DE (US); Robert DiCosimo, Chadds Ford, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/315,707

(22) Filed: Dec. 21, 2005

Related U.S. Application Data
(60) Provisional application No. 60/638,128, filed on Dec. 22, 2004.

(51) Int. Cl. C12P 7/40; C07C 51/42

(52) U.S. Cl. 435/136; 562/580

(57) ABSTRACT

The present invention relates to a method for producing glycolic acid from an aqueous solution of ammonium glycolate using reactive solvent extraction. More specifically, an aqueous solution of glycolic acid is created by acidifying an aqueous ammonium glycolate solution. An organic extraction solution comprising a tertiary trialkylamine is contacted with the aqueous glycolic acid solution, whereby the glycolic acid is extracted into the organic phase. A back extraction process is subsequently used to isolate the substantially purified glycolic acid from the organic phase.

This application claims the benefit of U.S. Provisional Application No. 60/638128, filed Dec. 22, 2004.

### FIELD OF THE INVENTION

This invention relates to a process for preparing glycolic acid from an aqueous solution of ammonium glycolate. More specifically, reactive solvent extraction is used to obtain glycolic acid from an aqueous solution of ammonium glycolate.

### BACKGROUND OF THE INVENTION

Glycolic acid (HOCH₂COOH; CAS Registry Number is 79-14-1) is the simplest member of the α-hydroxy acid family of carboxylic acids. Its properties make it ideal for a broad spectrum of consumer and industrial applications, including use in water well rehabilitation, the leather industry, the oil and gas industry, the laundry and textile industry, and as a component in personal care products like skin creams. Glycolic acid also is a principal ingredient for cleaners in a variety of industries (dairy and food processing equipment cleaners, household and institutional cleaners, industrial cleaners [for transportation equipment, masonry, printed circuit boards, stainless steel boiler and process equipment, cooling tower/heat exchangers], and metals processing [for metal pickling, copper brightening, etching, electroplating, electropolishing]). New technology to commercially produce glycolic acid would be eagerly received by industry.

Various methods for preparing α-hydroxy acids are known. Fermentative production of an α-hydroxy acid using the corresponding α-hydroxy nitrile as the starting material is known in the art. Examples of α-hydroxy acids produced via fermentation include: glycolic acid, lactic acid, 2-hydroxyisobutyric acid, 2-hydroxy-2-phenyl propionic acid, mandelic acid, 2-hydroxy-3,3-dimethyl-4-butyrolactone, and 4-methylthiobutyric acid. These products are synthesized using microorganisms, such as those belonging to the genera Nocardia, Bacillus, Brevibacterium, Aureobacterium, Pseudomonas, Caseobacter, Alcaligenes, Acinetobacter, Enterobacter, Arthrobacter, Escherichia, Micrococcus, Streptomyces, Flavobacterium, Aeromonas, Mycoplana, Cellulomonas, Erwinia, Candida, Bacteridium, Aspergillus, Penicillium, Cochliobolus, Fusarium, Rhodopseudomonas, Rhodococcus, Corynebacterium, Microbacterium, Obsumbacterium and Gordona. (JP-A-4-99495, JP-A-4-99496 and JP-A-4-218385 corresponding to U.S. Pat. No. 5,223,416; JP-A4-99497 corresponding to U.S. Pat. No. 5,234,826; JP-A-5-95795 corresponding to U.S. Pat. No. 5,296,373; JP-A-5-21987; JP-A-5-192189 corresponding to U.S. Pat. No. 5,326,702; JP-A-6-237789 corresponding to EP-A-0610048; JP-A-6-284899 corresponding to EP-A-0610049; JP-A-7-213296 corresponding to U.S. Pat. No. 5,508,181)

Acidovorax facilis 72W (ATCC 55746) is characterized by aliphatic nitrilase (EC 3.5.5.7) activity, as well as a combination of nitrile hydratase (EC 4.2.1.84) and amidase (EC 3.5.1.4) activities. The gene encoding the A. facilis 72W (ATCC 55746) nitrilase has been cloned and recombinantly expressed (WO 01/75077 corresponding to U.S. Pat. No. 6,870,038 and Chauhan et al., Appl Microbiol Biotechnol, 61:118-122 (2003)). The A. facilis 72W nitrilase converts α-hydroxynitriles to the corresponding α-hydroxycarboxylic acids in high yield (U.S. Pat. No. 6,383,786), including glycolic acid (U.S. Pat. No. 6,416,980).

Enzymatic conversion of glycolonitrile to glycolic acid using an enzyme catalyst (nitrilase or a combination of a nitrile hydratase and an amidase) typically results in the production of an aqueous solution of the ammonium glycolate. Many different methods have been suggested to convert the ammonium salt to free acid, such as the addition of a strong acid such as sulfuric acid (“acidification”). The glycolic acid thus obtained may be isolated by procedures such as concentration, precipitation, non-reactive solvent extraction, ion exchange, electrodialysis, thermal decomposition, distillation, and crystallization, to name a few. However, many of these conventional methods have limitations that are undesirable for industrial production such as 1) the use of low ammonium concentrations, 2) the generation of undesirable waste streams, 3) the use of expensive and difficult to operate processes (e.g., electrodialysis), or 4) the use of excessive amounts of energy. Conventional non-reactive solvent extraction is not a commercially viable option as the distribution coefficient for extracting the carboxylic acid into the organic phase may be very inefficient.

One method that has been used to isolate carboxylic acids is reactive extraction. This method has been reported to be useful for extracting lactic acid from ammonium lactate (Wasewar et al., J. Biotechnol., 97:59-68 (2002)). Reactive extraction involves the use of a reactive organic solvent (i.e., an amine) to complex with the acid in the aqueous phase. The first step in the process typically involves acidification of the aqueous solution containing the salt of the desired acid. The acidified aqueous solution is then contacted with an organic solvent typically comprised of a reactive tertiary amine and one or more diluents. The reactive amine (typically a tertiary C8-C10 trialkylamine such as Alamine® 336, Cognis Corp, Cincinnati, Ohio) reacts with the carboxylic acid forming an acid/amine complex that is preferentially soluble in the organic phase (Tamada et al., Ind. Eng. Chem. Res. 29:1319-1326 (1990); Tamada et al., Ind. Eng. Chem. Res. 29:1327-1333 (1990)). The use of a tertiary alkylamine typically provides much higher distribution coefficients than would be obtainable with normal solvent extraction. Back extraction is then used to recover the acid from the organic phase.

Inci, I. (Chem. Biochem. Eng. Q., 16(2):81-85 (2002); Inci, I. and Uslu, H., J. Chem. Eng. Data, 50:536-540 (2005)) report the use of reactive amine solvents for the extraction of glycolic acid. However, these experiments reported the extraction coefficients of pure glycolic acid dissolved in pure water. Inci does not illustrate or teach a process to obtain glycolic acid from a complex aqueous matrix (e.g., aqueous solutions of glycolic acid comprising significant amounts of mineral salts and other impurities), such as concentrated aqueous solutions of ammonium glycolate.

The problem to be solved is the lack of a process for obtaining glycolic acid from an aqueous solution of ammonium glycolate.

### SUMMARY OF THE INVENTION

The present problem has been solved by providing a process for obtaining glycolic acid from ammonium glycolate comprising

(a) providing a first phase, wherein said first phase is a water-immiscible organic solvent mixture comprising:
(i) about 30 volume percent to about 99 volume percent of said first phase is at least one tertiary alkyl amine having the formula 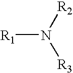
   wherein R₁, R₂, and R₃ are independently a C8 to C12 alkyl group; and
(ii) about 1 volume percent to about 70 volume percent of said first phase is at least one diluent selected from the group consisting of methyl isobutyl ketone, 1-octanol, 1-decanol, methylene chloride, 1-chlorobutane, chlorobenzene, chloroform, kerosene, toluene, mixed xylenes, tributyl phosphate, and mixtures thereof;

(b) providing a second phase, wherein said second phase is an aqueous solution comprising glycolic acid having a pH of about 3 or less; said second phase formed by the process of:
(i) providing an aqueous solution of ammonium glycolate; said aqueous solution of ammonium glycolate having a concentration about 5 weight % to about 40 weight % ammonium glycolate; and
(ii) adding an amount of mineral acid sufficient to lower the pH of the aqueous ammonium glycolate solution of (b)(i) to about 3 or less; whereby an aqueous solution comprising glycolic acid is formed;
(c) contacting said first phase with said second phase in a reactive extraction process; thereby forming a glycolic acid-loaded first phase;
(d) isolating said glycolic acid-loaded first phase;
(e) contacting said glycolic acid-loaded first phase with a third phase in a back extraction process; whereby glycolic acid in the glycolic acid-loaded first phase is extracted into said third phase; wherein said third phase is an aqueous solution that is immiscible in said glycolic acid-loaded first phase; and
(f) isolating the glycolic acid from said third phase.

### DETAILED DESCRIPTION OF THE INVENTION

The stated problem has been solved by providing an easy and efficient method to obtain glycolic acid from an aqueous solution of ammonium glycolate. More specifically, an aqueous solution of ammonium glycolate is first acidified. The resulting glycolic acid is extracted from the aqueous phase using reactive solvent extraction. The organic solvent is comprised of a tertiary amine and one or more diluents. The tertiary trialkyl amine (C8 to C12 alkyl groups) reacts with the glycolic acid, forming a glycolic acid:trialkylamine complex that is soluble in the organic phase. Back extraction into a second aqueous phase (“third phase”) is used to recover the acid from the glycolic acid-loaded organic phase.

Enzymatic conversion of an α-hydroxynitrile (e.g., glycolonitrile) to the corresponding α-hydroxyacid (e.g., glycolic acid) is well-known in the art. A nitrilase enzyme directly converts an aliphatic nitrile to the corresponding carboxylic acid, without forming the corresponding amide as intermediate (Equation 1). A particularly useful and robust catalyst (Acidovorax facilis 72W; ATCC 55746) has been used to convert glycolonitrile to glycolic acid in high yield (U.S. Pat. No. 6,416,980).

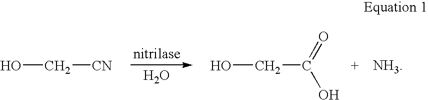

Enzymatic conversion of a nitrile to an acid typically results in the production of an aqueous solution of the ammonium salt of the carboxylic acid (e.g., ammonium glycolate) as the reaction conditions are usually maintained at a pH where the predominant species is the ammonium salt of the desired acid (pH typically about 6 to about 8). A variety of methods can used to convert the ammonium salt of the acid into the purified carboxylic acid including, but not limited to techniques based on concentration, crystallization, ion exchange (cationic and/or anionic), eletrodialysis, thermal decomposition of the salt, distillation, and alcoholysis.

The present method uses reactive solvent extraction to obtain an aqueous solution glycolic acid from an aqueous solution of ammonium glycolate. The aqueous solution of glycolic acid obtained by the present process has significantly fewer impurities (mineral salts, etc.). The substantially purified glycolic acid can be easily isolated using a variety of techniques known in the art.

The first step of the process involves the acidification of an aqueous ammonium glycolate solution to an aqueous solution that is predominantly glycolic acid. Typically, a mineral acid (e.g., H₂SO₄) is added to the aqueous ammonium glycolate solution until the pH of the solution is about 3 or less (the pKa of glycolic acid is ˜3.83). The acidified aqueous solution is then contacted with a water-immiscible organic solvent solution (“first phase”) comprised of a tertiary trialkylamine and at least one diluent selected from the group consisting of methyl isobutyl ketone, 1-octanol, 1-decanol, methylene chloride, 1-chlorobutane, chlorobenzene, chloroform, kerosene, toluene, mixed xylenes, and tributyl phosphate. The tertiary trialkylamine (e.g., Alamine® 336) reacts with the acid, forming an acid/amine complex that is soluble in the organic phase. Back extraction with water is then used to produce a substantially purified aqueous solution of glycolic acid. Methods to isolate the substantially purified glycolic acid obtained after back extraction are well known in the art and may include, but are not limited to crystallization, ion exchange, eletrodialysis, and distillation. Optionally, the organic solvent phase is recycled.

Definitions:

In this disclosure, a number of terms and abbreviations are used. The following definitions apply unless specifically stated otherwise.

As used herein, the term “comprising” means the presence of the stated features, integers, steps, or components as referred to in the claims, but that it does not preclude the presence or addition of one or more other features, integers, steps, components or groups thereof.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably with 5% of the reported numerical value.

“ATCC” refers to the American Type Culture Collection International Depository Authority located at ATCC, 10801 University Blvd., Manassas, Va. 20110-2209, USA. The “International Depository Designation” is the accession number to the culture on deposit with ATCC.

As used herein, the term “glycolonitrile” is abbreviated as “GLN” and is synonymous with hydroxyacetonitrile, 2-hydroxyacetonitrile, hydroxymethylnitrile, and all other synonyms of CAS Registry Number 107-164.

As used herein, the term “glycolic acid” is abbreviated as “GLA” and is synonymous with hydroxyacetic acid, hydroxyethanoic acid, and all other synonyms of CAS Registry Number 79-14-1.

As used herein, the term “ammonium” refers to the cation having the formula NH₄⁺.

As used herein, the term “ammonium glycolate” is the ammonium salt of glycolic acid and is abbreviated as “NH₄GLA:”

As used herein, the terms “Acidovorax facilis” and “A. facilis” are used interchangeably and refer to Acidovorax facilis 72W (ATCC 55746). The A. facilis 72W nitrilase is a particular robust catalyst that converts α-hydroxynitriles into the corresponding ammonium salt of the α-hydroxyacid using typical reaction conditions (U.S. Pat. No. 6,416,980; hereby incorporated by reference in its entirety).

As used herein, the terms “water-immiscible organic solvent” and “first phase” are used to describe an organic solvent mixture comprising at least one tertiary trialkylamine having the formula:

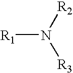

wherein R₁, R₂, and R₃ are independently a C8 to C12 alkyl group; and at least one diluent selected from the group consisting of methyl isobutyl ketone, 1-octanol, 1-decanol, methylene chloride, 1-chlorobutane, chlorobenzene, chloroform, kerosene, toluene, mixed xylenes, tributyl phosphate, and mixtures thereof. In one embodiment, the alkyl groups on the trialkylamine are independently C8 to C10 alkyl groups. In another embodiment, the tertiary trialkylamine is selected from the group consisting of tri-n-octylamine, tri-isooctylamine, tri-n-nonylamine, tri-n-decylamine, and tri-n-dodecylamine. In a further embodiment, the tertiary trialkylamine is selected from the group consisting of Alamine® 308 (CAS# 2757-28-0), Alamine® 300 (CAS# 1116-76-3), Alamine® 304-1 (CAS# 102-87-4), and Alamine® 336 (CAS# 68814-95-9) (Cognis Corp., Cincinnati, Ohio). In one embodiment, the diluent is selected from the group consisting of methyl isobutyl ketone (MIBK), kerosene, toluene, mixed xylenes, 1-octanol, and mixtures thereof. In another embodiment, the water-immiscible organic solvent is selected from the group consisting of 90% (vol/vol) Alamine® 336:10% (vol/vol) MIBK; 90% Alamine® 336:10% 1-octanol; 90% Alamine® 336:10% toluene; and 90% Alamine® 336:10% mixed xylenes.

The concentration of tertiary trialkyl amine in the first phase may range from about 30 percent (vol/vol) to about 99 percent (vol/vol), preferably about 50 percent (vol/vol) to about 90 percent (vol/vol), and most preferably about 70 percent (vol/vol) to about 90 percent (vol/vol). The amount of diluent in the first phase may range from about 1 percent (vol/vol) to about 70 percent (vol/vol), preferably about 10 percent to about 50 percent, and most preferably about 10 to about 30 percent.

As used herein, the term “aqueous solution of ammonium glycolate” will be used to describe the aqueous reaction mixture comprising ammonium glycolate (typically having a pH of about 6 to about 8).

As used herein, the term “second phase” refers to an aqueous solution of glycolic acid having a pH of about 4 or less, preferably about 3 or less, and most preferably about 1 to about 2. The “second phase” is prepared by adjusting the pH of the aqueous solution of ammonium glycolate with a strong mineral acid, such as H₂SO₄. However, the addition of the strong acid increases the amount of mineral salts (an undesirable impurity) in the second phase. Extraction of the glycolic acid from the second phase into an organic phase (i.e. the first phase) separates the glycolic acid from the mineral salt impurities.

As used herein, the term “reactive extraction process” refers to the process of contacting (i.e., mixing) an aqueous solution of glycolic acid (i.e., second phase) with a water-immiscible organic solvent (i.e., first phase) whereby the glycolic acid reacts with the tertiary trialkylamine to form an glycolic acid:trialkylamine complex. The complex is soluble in the organic phase, thereby extracting glycolic acid from the aqueous phase (i.e., second phase comprising substantial amounts of impurities) into the organic phase, forming a “glycolic acid-loaded first phase”. The glycolic acid-loaded first phase is subsequently isolated from the aqueous second phase. A back extraction process is then used to extract the glycolic acid from the organic phase back into an aqueous phase (i.e. the “third phase”). The length of time and temperature used for the reactive extraction process may be adjusted to optimize the extraction efficiency. In one embodiment, the mixing period of the first and second phase is about 5 minutes to about 8 hours, preferably about 5 minutes to about 1 hour, more preferably about 10 minutes to about 30 minutes. The temperature may range from about 5° C. to about 90° C., more preferably about 25° C. to about 75° C., and most preferably about 25° C. to about 50° C.

As used herein, the term “back extraction process” refers to the process of contacting a water-immiscible organic solvent comprising glycolic acid (i.e. “glycolic acid-loaded first phase”) with water (i.e. “third phase”) to extract the glycolic acid from the organic phase into the aqueous phase. In one embodiment, the third phase is deionized water. After back extraction, the third phase comprises a substantially purified form of glycolic acid (substantially less mineral salts and other impurities). The glycolic acid in the third phase can be optionally isolated using a variety of techniques know in the art. The length of time and temperature used for the back extraction process may be adjusted to optimize the extraction efficiency. In one embodiment, the mixing period of the “glycolic acid-loaded first phase” and aqueous phase (i.e., third phase) is about 10 minutes to about 8 hours, preferably about 30 minutes to about 4 hours, more preferably about 30 minutes to about 60 minutes. Typically, the back extraction process occurs under pressurized conditions under a non-reactive gas (i.e., nitrogen) blanket. The pressure in the back extraction chamber may be varied but is typically less than about 100 psig (less than about 690 kPa). The temperature may range from about 5° C. to about 150° C., preferably about 100° C. to about 150° C., and most preferably about 125° C. to about 140° C.

Suitable Conditions for the Present Process

Solvent extraction may also be used to obtain glycolic acid from an aqueous solution of ammonium glycolate. The concentration of ammonium glycolate in the aqueous solution is typically 5 wt % to about 90 wt %. In one embodiment, the concentration of ammonium glycolate is about 5 wt % to about 40 wt %. The aqueous solution of ammonium glycolate can comprise a reaction mixture resulting from the enzymatic hydrolysis of glycolonitrile that is unpurified or at least partially purified. The reaction mixture resulting from the enzymatic hydrolysis of glycolonitrile may also be comprised of other organic salts, inorganic salts, protein fragments, sugar residues, other organic acids, alcohols, ketones, and metal ions. The reaction mixture resulting from the enzymatic hydrolysis of glycolonitrile can be partially purified by filtration or centrifugation to remove excess debris or particulate matter that may result from the use of an unimmobilized cell or immobilized cell catalyst. In one embodiment, the feed stream may also be concentrated and/or acidified prior to being used as a feed stream in the present invention. The pH of the aqueous ammonium glycolate solution may range from about 5 to about 10, but is typically about 6 to about 8.

The first step in reactive solvent extraction is acidification (to a pH of about 4 or less, preferably a pH of about 0 to about 4, more preferably about 3 or less, and most preferably about 1 to about 2 of the aqueous ammonium glycolate solution with a strong acid, such a sulfuric acid (H₂SO₄). Lowering the pH of the ammonium glycolate solution creates an aqueous solution of glycolic acid (i.e., “second phase”) as the pKa of glycolic acid is about 3.83. This “second phase” typically is comprised of significant concentrations of ammonium and sulfate ions (undesirable impurities), creating a complex aqueous extract phase that may influence the overall reactive extraction process.

A water-immiscible organic solvent (i.e., “first phase”) is subsequently mixed with the second phase, and forms a two-phase system (a water immiscible organic phase and an aqueous phase). The glycolic acid in the second phase forms a complex with the tertiary trialkyl amine in the first phase. The complex has high solubility in the organic phase and low solubility in the aqueous phase, thereby extracting glycolic acid from the second (aqueous phase) into the first (organic phase), thereby forming a glycolic acid-loaded first phase. The partition coefficient of the glycolic acid can be calculated by measuring the wt % of the glycolic acid in the resulting loaded organic phase vs. the wt % of the remaining glycolic acid in the second phase.

A back extraction process is used to extract the glycolic acid from the glycolic acid-loaded first phase into a new aqueous phase (i.e., “third phase”). Typically, the glycolic acid-loaded first phase is isolated from the second phase (aqueous solution comprising glycolic acid and mineral salts). The loaded organic phase is subsequently contacted with a new aqueous phase (third phase). In one embodiment, the third phase is deionized water. During the back extraction process, glycolic acid from the organic phase is extracted back into an aqueous phase. The resulting aqueous solution of glycolic acid contains significantly fewer impurities compared to the water-soluble impurities (i.e., mineral salts) found in the acidified ammonium glycolate solution (i.e., second phase).

The substantially purified glycolic acid in the resulting aqueous phase can be isolated and purified using a variety of techniques well-known in the art including, but not limited to crystallization, distillation, etc.

Analytical Methods Use to Measure the Reactants and Products

A variety of analytical methods can be employed to analyze the reactants and products produced using the present methods include HPLC, GC, ion selective electrodes, MS, NMR, etc. HPLC was used to measure glycolic acid and various related products. Briefly, samples are diluted with water (as needed to be within HPLC detection range) and mixed 1:1 with 0.2 M n-propanol in water (HPLC internal standard). HPLC Analytical Method: (BioRad HPX 87H ion exclusion column (BioRad, Hercules, Calif.), 300 mm×7.8 mm; 0.01 N H₂SO₄ mobile phase; 1.0 mL/min flow at 50° C.; 10 μL injection volume; RI detector and UV 210 nm, 20 min analysis time). HPLC Equipment: (Waters 2695 ‘Alliance’ Separations Module, 2410 Refractive Index Detector, 2487 Dual λ Absorbance Detector and Empower Pro Software (Waters Corp, Milford, Mass.).

The following retention times were determined for various analytes using the above HPLC method (refractive index detector):

| Compound | RT min |
|---|---|
| Glycolic Dimer | 6.74 |
| Glycolic Acid | 7.65 |
| Glycolamide | 11.00 |
| n-propanol | 16.26 |

### General Methods

The following examples are provided to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Applicants specifically incorporate the entire contents of all cited references in this disclosure. Further, when an amount, concentration, or other value or parameter is given either as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The abbreviations in the specification correspond to units of measure, techniques, properties, or compounds as follows: “sec” means second(s), “min” means minute(s), “h” or “hr” means hour(s), “d” means day(s), “mL” means milliliters, “L” means liters, “mM” means millimolar, “M” means molar, “mmol” means millimole(s), “wt” means weight, “wt %” or “wt%” means weight percent, “g” means grams, “μg” means micrograms, “kPa” means kilopascal(s), and “HPLC” means high performance liquid chromatography.

### EXAMPLE 1

Solvent Extraction Using Approximately 70% C8-C10 Trialkylamine in Combination with 10% Methyl Isobutyl Ketone and 20% Kerosene at 25° C.

Into a 4-mL glass reactor equipped with magnetic stir bar was placed 1 mL of a mixed solvent containing 70% (volume/volume) of trialkyl amine (Alamine® 336; Cognis Corp., Cincinnati, Ohio), 10% (volume/volume) methyl isobutyl ketone (MIBK) and 20% (volume/volume) kerosene. The pH of an aqueous solution of ammonium glycolate (5 wt % to 40 wt %) was adjusted to about pH 2 to 3 with concentrated sulfuric acid (H₂SO₄), then 1 mL of the resulting aqueous solution was added to the reactor. The resulting mixture was stirred for 30 minutes at 25° C. The stirring was stopped and the two phases allowed to separate, then the organic and aqueous phases were each sampled and analyzed for glycolic acid concentration by HPLC. For each initial glycolic acid concentration, Table 1 lists the final concentration of glycolic acid in each phase of the resulting mixture, and the partition coefficient calculated for each initial glycolic acid concentration.

TABLE 1

| | | | Partition coefficient |
|---|---|---|---|
| Initial wt % | Final wt % | Final wt % | of glycolic acid |
| glycolic acid in | glycolic acid in | glycolic acid in | (organic wt %/ |
| aqueous phase | aqueous phase | organic phase | aqueous wt %) |
| 3.6 | 1.9 | 2.0 | 1.1 |
| 8.5 | 4.5 | 4.8 | 1.1 |
| 12.1 | 6.5 | 6.6 | 1.0 |
| 16.1 | 9.2 | 8.6 | 0.94 |
| 20.6 | 10.9 | 9.8 | 0.90 |
| 25.1 | 15.4 | 13.1 | 0.85 |
| 29.1 | 19.6 | 14.2 | 0.73 |
| 32.8 | 22.9 | 15.4 | 0.67 |

### EXAMPLE 2

Solvent Extraction Using Approximately 70% C8-C10 Trialkylamine in Combination with 10% Methyl Isobutyl Ketone and 20% Kerosene at 50° C.

Into a 4-mL glass reactor equipped with magnetic stir bar was placed 1 mL of a mixed solvent containing 70% (volume/volume) Alamine® 336 (Cognis), 10% (volume/volume) methyl isobutyl ketone (MIBK) and 20% (volume/volume) kerosene. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 50° C. The stirring was stopped and the two phases allowed to separate, then the organic and aqueous phases were each sampled and analyzed for glycolic acid concentration by HPLC. For each initial glycolic acid concentration, Table 2 lists the final concentration of glycolic acid in each phase of the resulting mixture, and the partition coefficient for each initial glycolic acid concentration.

TABLE 2

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 3.4 | 1.1 | 0.32 |
| 8.5 | 6.3 | 4.1 | 0.66 |
| 12.1 | 8.2 | 5.2 | 0.63 |
| 16.1 | 10.5 | 7.7 | 0.74 |
| 20.6 | 13.2 | 9.0 | 0.69 |
| 25.1 | 16.5 | 13.8 | 0.83 |
| 29.1 | 19.8 | 13.4 | 0.68 |
| 32.8 | 23.5 | 14.4 | 0.61 |

### EXAMPLE 3

Solvent Extraction Using Approximately 70% C8-C10 Trialkylamine in Combination with 10% Methyl Isobutyl Ketone and 20% Kerosene at 75° C.

Into a 4-mL glass reactor equipped with magnetic stir bar was placed 1 mL of a mixed solvent containing 70% (volume/volume) Alamine® 336 (Cognis), 10% (volume/volume) methyl isobutyl ketone (MIBK) and 20% (volume/volume) kerosene. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 75° C. The stirring was stopped and the two phases allowed to separate, then the organic and aqueous phases were each sampled and analyzed for glycolic acid concentration by HPLC. For each initial glycolic acid concentration, Table 3 lists the final concentration of glycolic acid in each phase of the resulting mixture, and the partition coefficient for each initial glycolic acid concentration.

TABLE 3

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 3.9 | 0.6 | 0.16 |
| 8.5 | 7.6 | 1.7 | 0.22 |
| 12.1 | 9.8 | 3.4 | 0.35 |
| 16.1 | 12.6 | 5.0 | 0.40 |
| 20.6 | 15.5 | 7.5 | 0.49 |
| 25.1 | 18.3 | 10.2 | 0.56 |
| 29.1 | 22.7 | 12.3 | 0.54 |
| 32.8 | 26.3 | 13.7 | 0.52 |

### EXAMPLE 4

Solvent Extraction Using Approximately 90% C8-C10 Trialkylamine in Combination with 10% Methyl Isobutyl Ketone at 25° C.

Into a 4-mL glass reactor equipped with magnetic stir bar was placed 1 mL of a mixed solvent containing 90% (volume/volume) Alamine® 336 (Cognis) and 10% (volume/volume) methyl isobutyl ketone (MIBK). The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 25° C. The stirring was stopped and the two phases allowed to separate, then the organic and aqueous phases were each sampled and analyzed for glycolic acid concentration by HPLC. For each initial glycolic acid concentration, Table 4 lists the final concentration of glycolic acid in each phase of the resulting mixture, and the partition coefficient for each initial glycolic acid concentration.

TABLE 4

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 1.8 | 3.6 | 1.98 |
| 8.5 | 4.3 | 6.8 | 1.57 |
| 12.1 | 5.9 | 8.1 | 1.38 |
| 16.1 | 8.4 | 12.2 | 1.44 |
| 20.6 | 12.6 | 14.2 | 1.13 |
| 25.1 | 13.6 | 16.2 | 1.19 |
| 29.1 | 16.6 | 18.9 | 1.14 |
| 32.8 | 21.1 | 19.4 | 0.92 |

### EXAMPLE 5

Solvent Extraction Using Approximately 90% C8-C10 Trialkylamine in Combination with 10% Methyl Isobutyl Ketone at 75° C.

Into a 4-mL glass reactor equipped with magnetic stir bar was placed 1 mL of a mixed solvent containing 90% (volume/volume) Alamine® 336 (Cognis) and 10% (volume/volume) methyl isobutyl ketone (MIBK). The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 75° C. The stirring was stopped and the two phases allowed to separate, then the organic and aqueous phases were each sampled and analyzed for glycolic acid concentration by HPLC. For each initial glycolic acid concentration, Table 5 lists the final concentration of glycolic acid in each phase of the resulting mixture, and the partition coefficient for each initial glycolic acid concentration.

TABLE 5

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 2.4 | 1.8 | 0.75 |
| 8.5 | 5.6 | 4.0 | 0.70 |
| 12.1 | 7.7 | 5.8 | 0.76 |
| 16.1 | 10.9 | 7.9 | 0.73 |
| 20.6 | 12.8 | 10.0 | 0.79 |
| 25.1 | 16.0 | 13.8 | 0.87 |
| 29.1 | 18.4 | 15.5 | 0.84 |
| 32.8 | 21.7 | 18.6 | 0.86 |

### EXAMPLE 6

Solvent Extraction Using Approximately 50% C8-C10 Trialkylamine in Combination with 10% Methyl Isobutyl Ketone and 40% Kerosene at 25° C.

Into a 4-mL glass reactor equipped with magnetic stir bar was placed 1 mL of a mixed solvent containing 50% (volume/volume) Alamine® 336 (Cognis), 10% (volume/volume) methyl isobutyl ketone (MIBK) and 40% (volume/volume) kerosene. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 25° C. The stirring was stopped and the two phases allowed to separate, then the organic and aqueous phases were each sampled and analyzed for glycolic acid concentration by HPLC. For each initial glycolic acid concentration, Table 6 lists the final concentration of glycolic acid in each phase of the resulting mixture, and the partition coefficient for each initial glycolic acid concentration.

TABLE 6

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 2.2 | 1.6 | 0.76 |
| 8.5 | 5.2 | 3.7 | 0.71 |
| 12.1 | 7.7 | 5.6 | 0.72 |
| 16.1 | 11.0 | 7.1 | 0.65 |
| 20.6 | 13.8 | 8.1 | 0.59 |
| 25.1 | 18.5 | 9.4 | 0.51 |
| 29.1 | 21.9 | 10.5 | 0.48 |
| 32.8 | 26.1 | 12.1 | 0.46 |

### EXAMPLE 7

Solvent Extraction Using Approximately 50% C8-C10 Trialkylamine in Combination with 10% Methyl Isobutyl Ketone and 40% Kerosene at 75° C.

Into a 4-mL glass reactor equipped with magnetic stir bar was placed 1 mL of a mixed solvent containing 50% (volume/volume) Alamine® 336 (Cognis), 10% (volume/volume) methyl isobutyl ketone (MIBK) and 40% (volume/volume) kerosene. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 75° C. The stirring was stopped and the two phases allowed to separate, then the organic and aqueous phases were each sampled and analyzed for glycolic acid concentration by HPLC. For each initial glycolic acid concentration, Table 7 lists the final concentration of glycolic acid in each phase of the resulting mixture, and the partition coefficient for each initial glycolic acid concentration.

TABLE 7

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 2.7 | 1.0 | 0.38 |
| 8.5 | 6.6 | 2.1 | 0.32 |
| 12.1 | 9.4 | 3.3 | 0.35 |
| 16.1 | 13.5 | 3.8 | 0.28 |
| 20.6 | 16.2 | 5.4 | 0.33 |
| 25.1 | 20.1 | 7.0 | 0.35 |
| 29.1 | 23.9 | 8.3 | 0.35 |
| 32.8 | 27.4 | 9.5 | 0.35 |

### EXAMPLE 8

Solvent Extraction Using Approximately 90% C8-C10 Trialkylamine in Combination with 10% 1-Octanol at 25° C.

Into a 4-mL glass reactor equipped with magnetic stir bar was placed 1 mL of a mixed solvent containing 90% (volume/volume) Alamine® 336 (Cognis), 10% (volume/volume) 1-octanol. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 25° C. The stirring was stopped and the two phases allowed to separate, then the organic and aqueous phases were each sampled and analyzed for glycolic acid concentration by HPLC. For each initial glycolic acid concentration, Table 8 lists the final concentration of glycolic acid in each phase of the resulting mixture, and the partition coefficient for each initial glycolic acid concentration.

TABLE 8

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 1.9 | 2.0 | 1.09 |
| 8.5 | 4.4 | 4.6 | 1.03 |
| 12.1 | 5.8 | 7.5 | 1.30 |
| 16.1 | 8.5 | 10.2 | 1.20 |
| 20.6 | 10.4 | 12.4 | 1.20 |
| 25.1 | 13.8 | 15.2 | 1.10 |
| 29.1 | 17.2 | 16.9 | 0.99 |
| 32.8 | 21.7 | 17.7 | 0.82 |

### EXAMPLE 9

Solvent Extraction Using Approximately 90% C8-C10 Trialkylamine in Combination with 10% 1-Octanol at 75° C.

Into a 4-mL glass reactor equipped with magnetic stir bar was placed 1 mL of a mixed solvent containing 90% (volume/volume) Alamine® 336 (Cognis), 10% (volume/volume) 1-octanol. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 75° C. The stirring was stopped and the two phases allowed to separate, then the organic and aqueous phases were each sampled and analyzed for glycolic acid concentration by HPLC. For each initial glycolic acid concentration, Table 9 lists the final concentration of glycolic acid in each phase of the resulting mixture, and the partition coefficient for each initial glycolic acid concentration.

TABLE 9

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 2.5 | 1.4 | 0.54 |
| 8.5 | 5.9 | 3.3 | 0.56 |
| 12.1 | 8.4 | 5.2 | 0.62 |
| 16.1 | 11.3 | 7.2 | 0.63 |
| 20.6 | 13.3 | 9.7 | 0.73 |
| 25.1 | 16.8 | 12.6 | 0.75 |
| 29.1 | 19.5 | 14.2 | 0.73 |
| 32.8 | 22.8 | 16.1 | 0.70 |

### EXAMPLE 10

Solvent Extraction Using Approximately 70% C8-C10 Trialkylamine in Combination with 30% 1-Octanol at 25° C.

Into a 4-mL glass reactor equipped with magnetic stir bar was placed 1 mL of a mixed solvent containing 70% (volume/volume) Alamine® 336 (Cognis), 30% (volume/volume) 1-octanol. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 25° C. The stirring was stopped and the two phases allowed to separate, then the organic and aqueous phases were each sampled and analyzed for glycolic acid concentration by HPLC. For each initial glycolic acid concentration, Table 10 lists the final concentration of glycolic acid in each phase of the resulting mixture, and the partition coefficient for each initial glycolic acid concentration.

TABLE 10

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 1.8 | 2.0 | 1.10 |
| 8.5 | 4.5 | 4.5 | 1.01 |
| 12.1 | 6.8 | 7.0 | 1.02 |
| 16.1 | 9.7 | 8.7 | 0.90 |
| 20.6 | 12.8 | 9.8 | 0.76 |
| 25.1 | 16.9 | 11.2 | 0.67 |
| 29.1 | 20.7 | 12.3 | 0.60 |
| 32.8 | 24.9 | 13.4 | 0.54 |

### EXAMPLE 11

Solvent Extraction Using Approximately 70% C8-C10 Trialkylamine in Combination with 30% 1-Octanol at 75° C.

Into a 4-mL glass reactor equipped with magnetic stir bar was placed 1 mL of a mixed solvent containing 70% (volume/volume) Alamine® 336 (Cognis), 30% (volume/volume) 1-octanol. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 75° C. The stirring was stopped and the two phases allowed to separate, then the organic and aqueous phases were each sampled and analyzed for glycolic acid concentration by HPLC. For each initial glycolic acid concentration, Table 11 lists the final concentration of glycolic acid in each phase of the resulting mixture, and the partition coefficient for each initial glycolic acid concentration.

TABLE 11

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 2.3 | 1.6 | 0.69 |
| 8.5 | 5.5 | 4.1 | 0.74 |
| 12.1 | 8.5 | 5.4 | 0.64 |
| 16.1 | 11.0 | 7.5 | 0.68 |
| 20.6 | 14.0 | 8.6 | 0.62 |
| 25.1 | 18.2 | 11.1 | 0.61 |
| 29.1 | 24.1 | 12.0 | 0.50 |
| 32.8 | 25.5 | 13.4 | 0.52 |

### EXAMPLE 12

Solvent Extraction Using Approximately 90% C8-C10 Trialkylamine in Combination with 10% Toluene at 25° C.

Into a 4-mL glass reactor equipped with magnetic stir bar was placed 1 mL of a mixed solvent containing 90% (volume/volume) Alamine® 336 (Cognis), 10% (volume/volume) toluene. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 25° C. The stirring was stopped and the two phases allowed to separate, then the organic and aqueous phases were each sampled and analyzed for glycolic acid concentration by HPLC. For each initial glycolic acid concentration, Table 12 lists the final concentration of glycolic acid in each phase of the resulting mixture, and the partition coefficient for each initial glycolic acid concentration.

TABLE 12

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 1.9 | 2.4 | 1.22 |
| 8.5 | 4.6 | 6.5 | 1.41 |
| 12.1 | 6.0 | 8.9 | 1.46 |
| 16.1 | 8.8 | 10.8 | 1.23 |
| 20.6 | 11.0 | 13.2 | 1.20 |
| 25.1 | 14.4 | 18.2 | 1.26 |
| 29.1 | 17.7 | 17.8 | 1.00 |
| 32.8 | 23.0 | 19.8 | 0.86 |

### EXAMPLE 13

Solvent Extraction Using Approximately 90% C8-C10 Trialkylamine in Combination with 10% Toluene at 75° C.

Into a 4-mL glass reactor equipped with magnetic stir bar was placed 1 mL of a mixed solvent containing 90% (volume/volume) Alamine® 336 (Cognis), 10% (volume/volume) toluene. The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 75° C. The stirring was stopped and the two phases allowed to separate, then the organic and aqueous phases were each sampled and analyzed for glycolic acid concentration by HPLC. For each initial glycolic acid concentration, Table 13 lists the final concentration of glycolic acid in each phase of the resulting mixture, and the partition coefficient for each initial glycolic acid concentration.

TABLE 13

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 2.6 | 1.3 | 0.51 |
| 8.5 | 6.0 | 3.5 | 0.58 |
| 12.1 | 8.3 | 5.5 | 0.67 |
| 16.1 | 11.9 | 7.5 | 0.63 |
| 20.6 | 13.8 | 9.0 | 0.65 |
| 25.1 | 16.4 | 12.0 | 0.73 |
| 29.1 | 19.3 | 14.5 | 0.75 |
| 32.8 | 22.0 | 16.6 | 0.75 |

### EXAMPLE 14

Solvent Extraction Using Approximately 90% C8-C10 Trialkylamine in Combination with 10% Xylenes at 25° C.

Into a 4-mL glass reactor equipped with magnetic stir bar was placed 1 mL of a mixed solvent containing 90% (volume/volume) Alamine® 336 (Cognis), 10% (volume/volume) xylenes (mixed xylene isomers). The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 25° C. The stirring was stopped and the two phases allowed to separate, then the organic and aqueous phases were each sampled and analyzed for glycolic acid concentration by HPLC. For each initial glycolic acid concentration, Table 14 lists the final concentration of glycolic acid in each phase of the resulting mixture, and the partition coefficient for each initial glycolic acid concentration.

TABLE 14

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 1.9 | 2.5 | 1.31 |
| 8.5 | 4.4 | 6.1 | 1.39 |
| 12.1 | 5.7 | 8.0 | 1.40 |
| 16.1 | 8.2 | 10.3 | 1.25 |
| 20.6 | 10.1 | 12.8 | 1.27 |
| 25.1 | 15.1 | 15.1 | 1.00 |
| 29.1 | 16.2 | 22.7 | 1.40 |
| 32.8 | 20.5 | 18.6 | 0.91 |

### EXAMPLE 15

Solvent Extraction Using Approximately 90% C8-C10 Trialkylamine in Combination with 10% Xylenes at 75° C.

Into a 4-mL glass reactor equipped with magnetic stir bar was placed 1 mL of a mixed solvent containing 90% (volume/volume) Alamine® 336 (Cognis), 10% (volume/volume) xylenes (mixed xylene isomers). The pH of an aqueous solution of ammonium glycolate (5 wt % to 40 wt %) was adjusted to pH 2 to 3 with concentrated sulfuric acid, then 1 mL of the resulting aqueous solution was added to the reactor. The resulting mixture was stirred for 30 minutes at 75° C. The stirring was stopped and the two phases allowed to separate, then the organic and aqueous phases were each sampled and analyzed for glycolic acid concentration by HPLC. For each initial glycolic acid concentration, Table 15 lists the final concentration of glycolic acid in each phase of the resulting mixture, and the partition coefficient for each initial glycolic acid concentration.

TABLE 15

| Initial wt % | Final wt % | Final wt % | |
|---|---|---|---|
| glycolic acid in | glycolic acid in | glycolic acid in | Partition coefficient |
| aqueous phase | aqueous phase | organic phase | (wt % org./wt % aq.) |
| 3.6 | 2.6 | 1.4 | 0.55 |
| 8.5 | 6.0 | 3.3 | 0.55 |
| 12.1 | 8.4 | 5.6 | 0.66 |
| 16.1 | 11.6 | 7.4 | 0.64 |
| 20.6 | 14.0 | 9.1 | 0.65 |
| 25.1 | 16.4 | 12.0 | 0.73 |
| 29.1 | 19.2 | 14.4 | 0.75 |
| 32.8 | 22.5 | 16.1 | 0.72 |

### EXAMPLE 16

Back Extraction Using Water from a Loaded Solvent of Approximately 70% C8-C10 Trialkylamine in Combination with 10% Methyl Isobutyl Ketone and 20% Kerosene

Following the procedures in Example 1, into a 1-L cylindrical glass vessel on an extraction mixer (mix by rotating back and forth vertically) was placed 100 mL of a mixed solvent containing 70% (volume/volume) Alamine® 336 (Cognis), 10% (volume/volume) methyl isobutyl ketone (MIBK) and 20% (volume/volume) kerosene. The pH of an aqueous solution of ammonium glycolate (10 wt % to 50 wt %) was adjusted to approximately pH 2 to 3 with concentrated sulfuric acid, then 100 mL of the resulting aqueous solution was added to the extraction mixer. The resulting mixture was stirred for 60 minutes at room temperature. The mixing was stopped and the two phases allowed to separate, then the organic and aqueous phases were each sampled and analyzed for glycolic acid concentration by HPLC. The organic phase was collected and used in the back extraction. This organic phase containing glycolic acid is referred as “loaded solvent” below.

Into a 85-mL pressure reaction glass tube (pressure reaction vessel, from Andrews Glass Co.) equipped with magnetic stir bar and double dip tubes was placed 10 mL deionized water and 10 mL of the loaded solvent. The vessel was then closed, and the headspace purged with nitrogen. The resulting mixture was stirred for 60 minutes at 120° C. under 40 psig (˜275.8 kPa) nitrogen. The stirring was stopped and the two phases allowed to separate at 120° C., then the organic phase was sampled under pressure through the top dip tube into a Hoke cylinder, and aqueous phases was sampled under pressure through the bottom dip tube into another Hoke cylinder. Both phases were analyzed for glycolic acid concentration by HPLC.

For each initial glycolic acid concentration in the loaded solvent, Table 16 lists the final concentration of glycolic acid in each phase of the resulting mixture, and the partition coefficient for each initial glycolic acid concentration.

TABLE 16

| Initial wt % | Final wt % | Final wt % | |
|---|---|---|---|
| glycolic acid in | glycolic acid in | glycolic acid in | Partition coefficient |
| loaded solvent | aqueous phase | organic phase | (wt % org./wt % aq.) |
| 3.9 | 1.7 | 0.82 | 0.47 |
| 11.3 | 6.5 | 3.4 | 0.53 |
| 16.0 | 9.0 | 4.6 | 0.51 |
| 17.5 | 9.8 | 5.1 | 0.52 |

### EXAMPLE 17

Back Extraction from a Loaded Solvent of Approximately 70% C8-C10 Trialkylamine in Combination with 10% Methyl Isobutyl Ketone and 20% Kerosene

Into a 85-mL pressure reaction glass tube (pressure reaction vessel, from Andrews Glass Co.) equipped with magnetic stir bar and double dip tubes was placed 10 mL aqueous solution of glycolic acid (20 wt % or 40 wt %) and 10 mL of the loaded solvent (see Example 16). The vessel was then closed, and the headspace purged with nitrogen. The resulting mixture was stirred for 60 minutes at 120° C. under 40 psig (˜275.8 kPa) nitrogen. The stirring was stopped and the two phases allowed to separate at 120° C, then the organic phase was sampled under pressure through the top dip tube into a Hoke cylinder, and aqueous phases was sampled under pressure through the bottom dip tube into another Hoke cylinder. Both phases were analyzed for glycolic acid concentration by HPLC.

For each initial glycolic acid concentration in the loaded solvent and aqueous solution, Table 17 lists the final concentration of glycolic acid in each phase of the resulting mixture, and the partition coefficient for each initial glycolic acid concentration.

TABLE 17

| Initial wt % | Initial wt % | Final wt % | Final wt % | |
|---|---|---|---|---|
| glycolic acid in | glycolic acid in | glycolic acid in | glycolic acid in | Partition coefficient |
| aqueous phase | loaded solvent | aqueous phase | organic phase | (wt % org./wt % aq.) |
| 20.0 | 16.0 | 20.7 | 12.1 | 0.59 |
| 40.0 | 17.5 | 33.7 | 17.3 | 0.51 |

### EXAMPLE 18

Back Extraction Using Water from a Loaded Solvent of Approximately 70% C8-C10 Trialkylamine in Combination with 10% Methyl Isobutyl Ketone and 20% Kerosene

Into a 85-mL pressure reaction glass tube (pressure reaction vessel, from Andrews Glass Co.) equipped with magnetic stir bar and double dip tubes was placed 10 mL deionized water and 10 mL of the loaded solvent (see Example 16). The vessel was then closed, and the headspace purged with nitrogen. The resulting mixture was stirred for 60 minutes at 140° C. under 40 psig (˜275.8 kPa) nitrogen. The stirring was stopped and the two phases allowed to separate at 140° C., then the organic phase was sampled under pressure through the top dip tube into a Hoke cylinder, and aqueous phases was sampled under pressure through the bottom dip tube into another Hoke cylinder. Both phases were analyzed for glycolic acid concentration by HPLC.

For each initial glycolic acid concentration in the loaded solvent, Table 18 lists the final concentration of glycolic acid in each phase of the resulting mixture, and the partition coefficient for each initial glycolic acid concentration.

TABLE 18

| Initial wt % | Final wt % | Final wt % | |
|---|---|---|---|
| glycolic acid in | glycolic acid in | glycolic acid in | Partition coefficient |
| loaded solvent | aqueous phase | organic phase | (wt % org./wt % aq.) |
| 3.9 | 2.8 | 1.35 | 0.48 |
| 11.3 | 6.2 | 2.3 | 0.37 |
| 16.0 | 9.4 | 3.2 | 0.34 |
| 17.5 | 10.2 | 3.6 | 0.35 |

### EXAMPLE 19

Back Extraction from a Loaded Solvent of Approximately 70% C8-C10 Trialkylamine in Combination with 10% Methyl Isobutyl Ketone and 20% Kerosene

Into a 85-mL pressure reaction glass tube (pressure reaction vessel, from Andrews Glass Co.) equipped with magnetic stir bar and double dip tubes was placed 10 mL aqueous solution of glycolic acid (20 wt % or 40 wt %) and 10 mL of the loaded solvent (see Example 16). The vessel was then closed, and the headspace purged with nitrogen. The resulting mixture was stirred for 60 minutes at 140° C. under 40 psig (˜275.8 kPa) nitrogen. The stirring was stopped and the two phases allowed to separate at 140° C., then the organic phase was sampled under pressure through the top dip tube into a Hoke cylinder, and aqueous phases was sampled under pressure through the bottom dip tube into another Hoke cylinder. Both phases were analyzed for glycolic acid concentration by HPLC.

For each initial glycolic acid concentration in the loaded solvent and aqueous solution, Table 19 lists the final concentration of glycolic acid in each phase of the resulting mixture, and the partition coefficient for each initial glycolic acid concentration.

TABLE 19

| Initial wt % | Initial wt % | Final wt % | Final wt % | |
|---|---|---|---|---|
| glycolic acid in | glycolic acid in | glycolic acid in | glycolic acid in | Partition coefficient |
| aqueous phase | loaded solvent | aqueous phase | organic phase | (wt % org./wt % aq.) |
| 20.0 | 16.0 | 21.9 | 11.7 | 0.54 |
| 40.0 | 17.5 | 34.4 | 18.7 | 0.54 |

### EXAMPLE 20

Back Extraction Using Water from a Loaded Solvent of Approximately 70% C8-C10 Trialkylamine in Combination with 30% Toluene

Into a 85-mL pressure reaction glass tube (pressure reaction vessel, from Andrews Glass Co.) equipped with magnetic stir bar and double dip tubes was placed 10 mL deionized water and 10 mL of the loaded solvent (see Example 16). The vessel was then closed, and the headspace purged with nitrogen. The resulting mixture was stirred for 60 minutes at 120° C. under 40 psig (˜275.8 kPa) nitrogen. The stirring was stopped and the two phases allowed to separate at 120° C., then the organic phase was sampled under pressure through the top dip tube into a Hoke cylinder, and aqueous phases was sampled under pressure through the bottom dip tube into another Hoke cylinder. Both phases were analyzed for glycolic acid concentration by HPLC.

For each initial glycolic acid concentration in the loaded solvent, Table 20 lists the final concentration of glycolic acid in each phase of the resulting mixture, and the partition coefficient for each initial glycolic acid concentration.

TABLE 20

| Initial wt % | Final wt % | Final wt % | |
|---|---|---|---|
| glycolic acid in | glycolic acid in | glycolic acid in | Partition coefficient |
| loaded solvent | aqueous phase | organic phase | (wt % org./wt % aq.) |
| 3.7 | 2.6 | 0.75 | 0.28 |
| 10.7 | 6.7 | 2.9 | 0.42 |
| 14.2 | 8.5 | 4.4 | 0.52 |
| 15.7 | 10.0 | 3.8 | 0.38 |

### EXAMPLE 21

Back Extraction from a Loaded Solvent of Approximately 70% C8-C10 Trialkylamine in Combination with 30% Toluene

Into a 85-mL pressure reaction glass tube (pressure reaction vessel, from Andrews Glass Co.) equipped with magnetic stir bar and double dip tubes was placed 10 mL aqueous solution of glycolic acid (20 wt % or 40 wt %) and 10 mL of the loaded solvent (see Example 16). The vessel was then closed, and the headspace purged with nitrogen. The resulting mixture was stirred for 60 minutes at 120° C. under 40 psig (˜275.8 kPa) nitrogen. The stirring was stopped and the two phases allowed to separate at 120° C., then the organic phase was sampled under pressure through the top dip tube into a Hoke cylinder, and aqueous phases was sampled under pressure through the bottom dip tube into another Hoke cylinder. Both phases were analyzed for glycolic acid concentration by HPLC.

For each initial glycolic acid concentration in the loaded solvent and aqueous solution, Table 21 lists the final concentration of glycolic acid in each phase of the resulting mixture, and the partition coefficient for each initial glycolic acid concentration.

TABLE 21

| Initial wt % | Initial wt % | Final wt % | Final wt % | |
|---|---|---|---|---|
| glycolic acid in | glycolic acid in | glycolic acid in | glycolic acid in | Partition coefficient |
| aqueous phase | loaded solvent | aqueous phase | organic phase | (wt % org./wt % aq.) |
| 20.0 | 14.2 | 21.9 | 11.7 | 0.54 |
| 40.0 | 15.7 | 34.4 | 18.7 | 0.54 |

### EXAMPLE 22

Back Extraction Using Water from a Loaded Solvent of Approximately 70% C8-C10 Trialkylamine in Combination with 30% Toluene

Into a 85-mL pressure reaction glass tube (pressure reaction vessel, from Andrews Glass Co.) equipped with magnetic stir bar and double dip tubes was placed 10 mL deionized water and 10 mL of the loaded solvent (see Example 16). The vessel was then closed, and the headspace purged with nitrogen. The resulting mixture was stirred for 60 minutes at 140° C. under 40 psig (˜275.8 kPa) nitrogen. The stirring was stopped and the two phases allowed to separate at 140° C., then the organic phase was sampled under pressure through the top dip tube into a Hoke cylinder, and aqueous phases was sampled under pressure through the bottom dip tube into another Hoke cylinder. Both phases were analyzed for glycolic acid concentration by HPLC.

For each initial glycolic acid concentration in the loaded solvent, Table 20 lists the final concentration of glycolic acid in each phase of the resulting mixture, and the partition coefficient for each initial glycolic acid concentration.

TABLE 22

| Initial wt % | Final wt % | Final wt % | |
|---|---|---|---|
| glycolic acid in | glycolic acid in | glycolic acid in | Partition coefficient |
| loaded solvent | aqueous phase | organic phase | (wt % org./wt % aq.) |
| 3.7 | 2.7 | 0.70 | 0.26 |
| 10.7 | 7.6 | 2.3 | 0.30 |
| 14.2 | 9.5 | 3.6 | 0.38 |
| 15.7 | 10.1 | 2.8 | 0.28 |

### EXAMPLE 23

Back Extraction from a Loaded Solvent of Approximately 70% C8-C10 Trialkylamine in Combination with 30% Toluene

Into a 85-mL pressure reaction glass tube (pressure reaction vessel, from Andrews Glass Co.) equipped with magnetic stir bar and double dip tubes was placed 10 mL aqueous solution of glycolic acid (20 wt % or 40 wt %) and 10 mL of the loaded solvent (see Example 16). The vessel was then closed, and the headspace purged with nitrogen. The resulting mixture was stirred for 60 minutes at 140° C. under 40 psig (˜275.8 kPa) nitrogen. The stirring was stopped and the two phases allowed to separate at 140° C., then the organic phase was sampled under pressure through the top dip tube into a Hoke cylinder, and aqueous phases was sampled under pressure through the bottom dip tube into another Hoke cylinder. Both phases were analyzed for glycolic acid concentration by HPLC.

For each initial glycolic acid concentration in the loaded solvent and aqueous solution, Table 23 lists the final concentration of glycolic acid in each phase of the resulting mixture, and the partition coefficient for each initial glycolic acid concentration.

TABLE 23

| Initial wt % | Initial wt % | Final wt % | Final wt % | |
|---|---|---|---|---|
| glycolic acid in | glycolic acid in | glycolic acid in | glycolic acid in | Partition coefficient |
| aqueous phase | loaded solvent | aqueous phase | organic phase | (wt % org./wt % aq.) |
| 20.0 | 14.2 | 23.2 | 10.2 | 0.44 |
| 40.0 | 15.7 | 37.4 | 16.9 | 0.45 |

What is claimed is:

1. A process for obtaining glycolic acid from an aqueous solution of ammonium glycolate comprising the steps of:
(a) providing a first phase, wherein said first phase is a water-immiscible organic solvent mixture comprising:
(i) about 30 volume percent to about 99 volume percent of said first phase is at least one tertiary alkyl amine having the formula 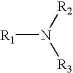
wherein R₁, R₂, and R₃ are independently a C8 to C12 alkyl group; and
(ii) about 1 volume percent to about 70 volume percent of said first phase is at least one diluent selected from the group consisting of methyl isobutyl ketone, 1-octanol, 1-decanol, methylene chloride, 1-chlorobutane, chlorobenzene, chloroform, kerosene, toluene, mixed xylenes, tributyl phosphate, and mixtures thereof;
(b) providing a second phase, wherein said second phase is an aqueous solution comprising glycolic acid having a pH of about 3 or less; said second phase formed by the process of:
(i) providing an aqueous solution of ammonium glycolate; said aqueous solution of ammonium glycolate having a concentration about 5 weight % to about 40 weight % ammonium glycolate; and
(ii) adding an amount of mineral acid sufficient to lower the pH of the aqueous ammonium glycolate solution of (b)(i) to about 3 or less;
whereby an aqueous solution comprising glycolic acid is formed;
(c) contacting said first phase with said second phase in a reactive extraction process; thereby forming a glycolic acid-loaded first phase;
(d) isolating said glycolic acid-loaded first phase;
(e) contacting said glycolic acid-loaded first phase with a third phase in a back-extraction process; whereby the glycolic acid in the glycolic acid-loaded first phase is extracted into said third phase; wherein said third phase is an aqueous solution that is immiscible in said glycolic acid-loaded first phase; and
(f) isolating the glycolic acid from said third phase.

2. The process of claim 1 wherein R₁, R₂, and R₃ are independently a C8 to C10 alkyl groups.

3. The process of claim 1 wherein the tertiary trialkylamine is selected from the group consisting of tri-isooctylamine, tri-n-octylamine, tri-n-nonylamine, tri-n-decylamine, tri-n-dodecylamine, and mixtures thereof.

4. The process of claim 2 wherein the tertiary trialkylamine is selected from the group consisting of Alamine® 300 having CAS# 1116-76-3, Alamine® 304-1 having CAS# 102-87-4, and Alamine® 336 having CAS# 68814-95-9.

5. The process of claim 4 wherein the tertiary trialkylamine is Alamine® 336.

6. The process of claim 4 or claim 5 wherein the first phase comprises about 50 percent to about 90 percent Alamine® 336.

7. The process of claim 1 wherein the diluent is selected from the group consisting of methyl isobutyl ketone, toluene, xylenes, 1-octanol, kerosene, and mixtures thereof.

8. The process of claim 7 wherein the first phases comprises about 10 percent to about 30 percent diluent.

9. The process of claim 1 wherein sulfuric acid is added to the aqueous solution of ammonium glycolate until the pH is about 2 to about 3.

10. The process of claim 1 wherein the reactive extraction process of step (1)(c) is performed at a temperature of about 25° C. to about 75° C.

11. The process of claim 10 wherein the back extraction process of step (1)(e) is performed at a temperature of about 120° C. to about 140° C.

12. The process of claim 11 wherein the back extraction process of step (1)(e) is performed at pressure of about 280 kPa or less.

13. The process of claim 1 wherein the aqueous solution comprising ammonium glycolate is provided by enzymatic conversion of glycolonitrile.
